(19) United States
(12) Patent Application Publication
(10) Pub. No.: US 2022/0054570 A1
(43) Pub. Date: Feb. 24, 2022

(54) COMPOSITION COMPRISING ELECAMPANE EXTRACT FOR ALLEVIATING PREMENSTRUAL SYNDROME SYMPTOM

(71) Applicant: GENENCELL INC., Gyeonggi-do (KR)

(72) Inventors: Se Chan Kang, Gyeonggi-do (KR); Yong Joon Jeong, Gyeonggi-do (KR); Yoon Jung Yang, Gyeonggi-do (KR)

(73) Assignee: GENENCELL INC., Gyeonggi-do (KR)

(21) Appl. No.: 17/298,745

(22) PCT Filed: Oct. 2, 2019
(86) PCT No.: PCT/KR2019/012938

§ 371 Date: Jun. 1, 2021

(30) Foreign Application Priority Data
Dec. 11, 2018 (KR) 10-2018-0159060

(51) Int. Cl. A61K 36/285; A61P 15/00

(52) U.S. Cl. CPC A61K 36/285; A61K 2236/331; A61K 2236/333; A61P 15/00

(57) ABSTRACT

The present invention relates to a composition comprising an elecampane extract for prevention and alleviation or treatment of premenstrual syndrome. In detail, the composition can be advantageously available as a composition for preventing, and alleviating or treating premenstrual syndrome in women, which effectively suppresses pituitary prolactin secretion, which is a phenomenon appearing in premenstrual syndrome, to increase the progesterone release amount lowered in the luteal phase of women as well as effectively controlling the decreased release of luteinizing hormone and follicle-stimulating hormone.

### TECHNICAL FIELD

The present invention relates to a composition for alleviating, preventing or treating premenstrual syndrome symptoms containing an elecampane extract as an active ingredient.

### BACKGROUND ART

The international classification of disease ([ICD]-10) defines “premenstrual syndrome” as a condition in which one or more of seven symptoms, namely, minor psychological discomfort, bloating, weight gain, breast tenderness, muscle tension or aches, poor concentration, and changes in appetite are exhibited, and these symptoms occur only in the luteal phase of the menstrual cycle. These symptoms begin to appear from 4 to 7 days before the onset of menstruation and disappear completely simultaneously with the onset of menstruation. Therefore, premenstrual syndrome is classified as a disease different from menstrual pain, which occurs when the endometrium peels off during the menstrual period.

The cause of premenstrual syndrome has not been clearly identified, but lifestyle habits such as hormonal imbalance, genetic propensity, ovulation and menstrual cycles, administration of drugs, smoking, alcohol and caffeine intake, dietary patterns, administration of contraceptives, emotional state, marital status, social factors, age, height, weight, birth, menstrual history, and stress are known to be related to premenstrual syndrome. In recent years, it has been reported that female hormones and progesterone, which change according to the menstrual cycle, may affect various neurotransmitters and lead to the occurrence of premenstrual syndrome. In particular, hormonal problems, such as a decrease in serotonin and an imbalance between progesterone and estrogen caused by an increase in prolactin, greatly affect the occurrence of premenstrual syndrome (Biggs and Demuth, 2011; Imai et al., 2015; Schmidt et al., 2017).

Prolactin is a milk-secretion-stimulating hormone secreted from coral cells in the anterior pituitary gland, and the amount thereof is known to be increased by estrogen and to be decreased by dopamine secreted from the hypothalamus. In normal subjects, the secretion of progesterone is higher than that of estrogen in the luteal phase, so the secretion of prolactin is stabilized. However, it has been reported that, in the case of women with premenstrual syndrome, the secretion of progesterone is lower than that of estrogen, so the secretion of prolactin is increased and the premenstrual syndrome is thus induced (Halbreich et al., 1976). Prefemin tablets (Vitex agnus-castus fruit extract), which exhibit an effect of inhibiting prolactin secretion based on this mechanism, have been marketed as a representative therapeutic agent for premenstrual syndrome, and clinical trials show that Prefemin tablets relieve about 50% of symptoms such as irritation, depression, anger, headaches, and breast pain.

Meanwhile, Aucklandiae radix has been used as a medicinal herb in China for a long time. A composition for preventing hair loss containing Aucklandiae radix as a raw material is disclosed in Korean Patent Laid-Open Publication No. 10-2015-0146981. In addition, a composition for ameliorating immune diseases using Aucklandiae radix and Thuja orientalis extracts is disclosed in Korean Patent Laid-Open Publication No. 10-2013-0118396. In addition, a composition for treating sterility and infertility using Aucklandiae radix is disclosed in Korean Patent Laid-Open Publication No. 10-2016-0151263.

However, no research on methods for preventing or treating premenstrual syndrome using Aucklandiae radix, a Korean traditional medicinal material, has been reported.

### PRIOR ART DOCUMENT

### Patent Document

(Patent Document 1) Korean Patent Laid-Open Publication No. 10-2015-0146981

(Patent Document 2) Korean Patent Laid-Open Publication No. 10-2013-0118396

(Patent Document 3) Korean Patent Laid-Open Publication No. 10-2016-0151263

(Patent Document 4) US Patent Laid-Open Publication No. 2018-0104295

(Patent Document 5) US Patent Laid-Open Publication No. 2016-0008406

### DISCLOSURE

### Technical Problem

Accordingly, the present inventors recognized the importance of prevention and treatment of female premenstrual syndrome, and selected natural resources that exhibit an effect of inhibiting secretion of prolactin in pituitary cells and an effect of proliferating pituitary cells using various natural materials. The result showed that the Aucklandiae radix extract strongly inhibited the secretion of prolactin in the pituitary gland cells. In addition, these compositions effectively regulate the secretion of progesterone in a hyperprolactinemia-inducing animal model, and thus have great potential for commercialization as compositions for preventing, ameliorating or treating premenstrual syndrome caused by the reversal of estrogen and progesterone.

Therefore, it is one object of the present invention to provide a pharmaceutical composition for preventing, ameliorating or treating premenstrual syndrome containing an Aucklandiae radix extract.

It is another object of the present invention to provide a health functional food composition for preventing and ameliorating premenstrual syndrome symptoms containing an Aucklandiae radix extract.

The objects of the present invention are not limited to those described above. The objects of the present invention will be clearly understood from the following description, and may be implemented by the means defined in the claims and combinations thereof.

### Technical Solution

In one aspect, the present invention provides a composition for preventing, ameliorating or treating premenstrual syndrome containing an Aucklandiae radix extract as an active ingredient.

In one aspect of the present invention, the composition may contain an index component of the Aucklandiae radix.

In one aspect of the present invention, the Aucklandiae radix may be Aucklandia lappa Decne. or Inula helenium L.

In one aspect of the present invention, the extract may be a water extract, a C₁-C₅ alcohol extract, or a C₁-C₅ alcohol aqueous solution extract.

In one aspect of the present invention, the Aucklandiae radix extract may be present in an amount of 0.001 to 90% by weight based on the total weight of the composition.

In one aspect of the present invention, the Aucklandiae radix extract may be a 10% to 80% ethanol aqueous solution extract.

In one aspect of the present invention, the composition may suppress the secretion of prolactin or increase the secretion of progesterone in a woman in the luteal phase before menstruation.

In one aspect of the present invention, the symptoms of premenstrual syndrome may include at least one of minor psychological discomfort, bloating, weight gain, breast tenderness, muscular tension or aches, poor concentration and changes in appetite, and the symptom of premenstrual syndrome may appear only in the luteal phase of the menstrual cycle.

In one aspect of the present invention, the composition for preventing or treating premenstrual syndrome may be a pharmaceutical composition.

In one aspect of the present invention, the composition for ameliorating premenstrual syndrome may be a health functional food composition.

### Advantageous Effects

The premenstrual syndrome extract contained as an active ingredient in the composition of the present invention has effects of significantly inhibiting the secretion of prolactin from the pituitary gland cells, increasing the secretion of progesterone in the luteal phase, and increasing the secretion of lowered luteinizing hormone and follicle-stimulating hormone. Therefore, the Aucklandiae radix extract can ameliorate, prevent, or treat a disease for which it is required to inhibit the secretion of prolactin and to increase the secretion of progesterone, that is, premenstrual syndrome.

The effects of the present invention are not limited to those mentioned above. It should be understood that the effects of the present invention include all effects that can be inferred from the description of the present invention.

### BRIEF DESCRIPTION OF DRAWINGS

FIG. 1 shows the effect of inhibiting prolactin secretion in a hyperprolactinemia-induced animal model by the Aucklandiae radix extract of the present invention.

FIG. 2 shows the effect of increasing the secretion of progesterone compared to estrogen in the hyperprolactinemia-induced animal model by the Aucklandiae radix extract of the present invention.

FIG. 3 shows the effect of increasing the secretion of luteinizing hormone (LH) in the hyperprolactinemia-induced animal model by the Aucklandiae radix extract of the present invention.

FIG. 4 shows the effect of increasing the secretion of follicle-stimulating hormone (FSH) in the hyperprolactinemia-induced animal model by the Aucklandiae radix extract of the present invention.

FIG. 5 shows the effect of inhibiting the secretion of prostaglandin E2 in the hyperprolactinemia-induced animal model by the Aucklandiae radix extract of the present invention.

### BEST MODE

Unless the context clearly indicates otherwise, all numbers, figures and/or expressions that represent ingredients, reaction conditions, polymer compositions and amounts of mixtures used in the specification are approximations that reflect various uncertainties of measurement occurring inherently in obtaining these figures, among other things. For this reason, it should be understood that, in all cases, the term “about” should modify all numbers, figures and/or expressions. In addition, when numerical ranges are disclosed in the description, these ranges are continuous and include all numbers from the minimum to the maximum, including the maximum within the range, unless otherwise defined. Furthermore, when the range refers to an integer, it includes all integers from the minimum to the maximum, including the maximum within the range, unless otherwise defined.

It should be understood that, in the specification, when a range is referred to regarding a parameter, the parameter encompasses all figures including end points disclosed within the range. For example, the range of “5 to 10” includes figures of 5, 6, 7, 8, 9, and 10, as well as arbitrary sub-ranges, such as ranges of 6 to 10, 7 to 10, 6 to 9, and 7 to 9, and any figures, such as 5.5, 6.5, 7.5, 5.5 to 8.5 and 6.5 to 9, between appropriate integers that fall within the range. In addition, for example, the range of “10% to 30%” encompasses all integers that include numbers such as 10%, 11%, 12% and 13%, as well as 30%, and any sub-ranges, such as 10% to 15%, 12% to 18%, or 20% to 30%, as well as any numbers, such as 10.5%, 15.5% and 25.5%, between appropriate integers that fall within the range.

Hereinafter, the present invention will be described in detail.

The present invention relates to a composition for preventing, ameliorating or treating premenstrual syndrome containing an Aucklandiae radix extract. Specifically, the composition effectively inhibits the secretion of prolactin from the pituitary gland cells, which is a phenomenon that occurs in premenstrual syndrome, to increase the secretion of progesterone, the amount of which is reduced during the female luteal phase, as well as the secretion of luteinizing hormone and follicle-stimulating hormone, thus being useful as a composition for preventing and ameliorating or treating premenstrual syndrome.

Aucklandiae radix (Aucklandia lappa Decne. or Inula helenium L.) is a perennial herb belonging to the Asteraceae family, and the root thereof is used as a medicinal herb. Aucklandiae radix acts as a blood circulation agent for promoting energy cycling and has analgesic, aromatic stomachic, and apophlegmatic activities and thus has been used in herbal or folk remedies for stomach cramps, vomiting, diarrhea, abdominal pain, indigestion, dyspepsia, ascariasis, stomach strengthening, dieresis, discharge of phlegm, sweating, asthma, bronchitis, tussis, tumescence, detoxification and the like. Reported major ingredients of Aucklandiae radix include sesquiterpene lactone compounds such as costunolide and dehydrocostus lactone, alantolactone, dihydrocostunolide, isodihydrocostunolide, isoalantolactone, mokko lactone, lignin, alkaloid, tannin, dihydrocostunolide, costus lactone, α-costol, saussurea lactone, and the like. In the Korean Herbal Pharmacopoeia and the Chinese Pharmacopoeia, the index components of Aucklandiae radix are defined as costunolide and dehydrocostus lactone, and the total content thereof is stipulated to be 1.8% or more.

The Aucklandiae radix extract has been reported to have various physiological activities such as anti-inflammatory, anticancer, antioxidant, antibacterial and anti-allergic activities. However, no literature published to date has revealed that the Aucklandiae radix extract and main components thereof are effective in preventing, ameliorating or treating female premenstrual syndrome by inhibiting the secretion of prolactin.

The present invention provides a pharmaceutical composition for preventing, ameliorating or treating premenstrual syndrome containing an Aucklandiae radix extract. The composition may be a liquid extract or a lyophilized powder of the liquid extract.

In addition, the present invention provides a health functional food composition for ameliorating premenstrual syndrome containing an Aucklandiae radix extract. The composition may be a liquid extract or a lyophilized powder of the liquid extract.

Hereinafter, various aspects of the present invention will be described.

In one aspect, the present invention provides a pharmaceutical composition for preventing, ameliorating or treating premenstrual syndrome containing an Aucklandiae radix extract as an active ingredient.

These compositions effectively inhibit the secretion of prolactin from the pituitary gland cells and increase the amount of progesterone secreted in the luteal phase. In addition, because the constituents thereof are natural products that have been conventionally ingested and administered on a daily basis, the compositions can be safely used for preventing, ameliorating or treating premenstrual syndrome.

The Aucklandiae radix extract may be extracted from Aucklandiae radix by a conventional extraction method, and the extract may be a powder acquired through reduced-pressure drying and freeze-drying.

The Aucklandiae radix may be Aucklandia lappa Decne. or Inula helenium L.

In one aspect of the present invention, the extract is a water extract, a C₁-C₅ alcohol extract or a C₁-C₅ alcohol aqueous solution extract.

The content of alcohol in the extraction solvent is 0 to 95% by weight, preferably 10 to 70% by weight, and more preferably 30% by weight, 50% by weight, or 70% by weight, in terms of efficiency. Any alcohol may be applied, as long as it is one that is generally used in the art, the alcohol is preferably C₁-C₅ alcohol, and the alcohol may include at least one selected from methanol, ethanol, isopropanol, propanol and butanol, and is more preferably ethanol or the like.

The extraction method is a method commonly known in the art, for example, a method using an extraction device of supercritical extraction, subcritical extraction, high-temperature extraction, high-pressure extraction, ultrasonic extraction or the like, or using an adsorption resin including XAD and HP-20.

In addition, the extract is obtained by concentration under reduced pressure using a vacuum rotary evaporator or the like. In addition, the obtained extract may be subjected to drying under reduced pressure, vacuum drying, boiling drying, spray drying, room-temperature drying, or freeze drying, if necessary. In particular, the freeze-drying method has the advantage of reducing the loss of volatile organic substances from the extract.

The Aucklandiae radix extract obtained through the above method effectively inhibits the secretion of prolactin from pituitary cells and increases the secretion of progesterone in the luteal phase. In addition, the composition containing the Aucklandiae radix extract obtained through the above method more effectively controls the secretion of prolactin and the secretion of progesterone from the pituitary gland cells.

In one aspect of the present invention, the Aucklandiae radix extract is present in an amount of 0.001 to 90% by weight based on the total weight of the composition.

In one aspect of the present invention, the Aucklandiae radix extract is a 10% to 80% ethanol aqueous solution extract.

In one aspect of the present invention, the composition suppresses the secretion of prolactin or increases the secretion of progesterone in a woman in the luteal phase before menstruation.

In one aspect of the present invention, a symptom of the premenstrual syndrome includes at least one of minor psychological discomfort, bloating, weight gain, breast tenderness, muscular tension or aches, poor concentration, and changes in appetite, and the symptoms of premenstrual syndrome appear only in the luteal phase of the menstrual cycle.

In one aspect of the present invention, the composition for preventing or treating premenstrual syndrome is a pharmaceutical composition.

When the pharmaceutical composition is used clinically, it is prepared into any of common formulations in the pharmaceutical field, for example, formulations for oral administration such as tablets, capsules, powders, granules, pills, liquids, and suspensions, injectable preparations such as solutions or suspensions for injection, or ready-to-use dry powders for injection that can be prepared using distilled water for injection at the time of injection, or various formulations such as ointments, by mixing the pharmaceutical composition with a conventional carrier in the pharmaceutical field. Pharmaceutical formulations prepared using conventional carriers can be administered orally or parenterally, for example, intravenously, subcutaneously, intraperitoneally or topically. Accordingly, the pharmaceutical composition of the present invention may further include an appropriate carrier, excipient, and diluent commonly used in the preparation of a drug.

The carrier, excipient and diluent that can be included in the pharmaceutical compositions of the present invention may include at least one selected from the group consisting of lactose, dextrose, sucrose, sorbitol, mannitol, xylitol, erythritol, maltitol, starch, gum acacia, alginate, gelatin, calcium phosphate, calcium silicate, cellulose, methyl cellulose, hydroxymethyl cellulose, microcrystalline cellulose, silicified microcrystalline cellulose, povidone, crospovidone, croscarmellose sodium, polyvinylpyrrolidone, water, methylhydroxybenzoate, propylhydroxybenzoate, Neusilin®, colloidal silicon dioxide, lactose, talc, magnesium stearate, colloidal stearyl magnesium, and mineral oil.

The formulation is prepared using diluents or excipients such as fillers, extenders, binders, wetting agents, disintegrants and surfactants that are commonly used in the art. Solid formulations for oral administration include tablets, pills, powders, granules, troches, lozenges, capsules, and the like, and these solid formulations are prepared by mixing the composition of the present invention with at least one excipient such as lactose, saccharose, sorbitol, mannitol, starch, amylopectin, cellulose, calcium carbonate, gelatin, or the like. In addition to simple excipients, lubricants such as magnesium stearate and talc are also used. Liquid formulations for oral administration include suspensions, oral liquids, emulsions, elixirs, and syrups. In addition to water and liquid paraffin, which are commonly used simple diluents, various excipients such as wetting agents (humectants), sweeteners, fragrances, preservatives, and the like may be included. Formulations for parenteral administration include sterilized aqueous solutions, non-aqueous solvents, suspensions, emulsions, lyophilized formulations, and suppositories. As the non-aqueous solvents and suspensions, propylene glycol, polyethylene glycol, vegetable oil such as olive oil, and injectable ester such as ethyl oleate may be used. As a base for suppositories, Witepsol, macrogol, Tween 61, cacao butter, laurin butter, glycerol gelatin, and the like may be used. Parenteral administration may generally be subcutaneous injection, intravenous injection, intramuscular injection, or intrathoracic injection.

The preferred dose of the pharmaceutical compound according to the present invention may vary depending on the age and weight of a patient, disease severity, drug type, and route and duration of administration, but may be appropriately selected by those skilled in the art. However, for desirable effects, the pharmaceutical composition of the present invention may be administered at 0.01 mg/kg to 10 g/kg per day, preferably 1 mg/kg to 1 g/kg. The composition may be administered in a portionwise manner one or more times, preferably one or six times, daily at a predetermined time interval according to the determination of a physician or pharmacist.

In another aspect, the composition for ameliorating premenstrual syndrome is a health functional food composition.

The health functional food composition according to the present invention contains the Aucklandiae radix extract as an active ingredient. The active ingredient may be ingested as a food prepared in the form of a formulation such as a tablet, capsule, powder, granule, pill, liquid, suspension or the like, or may be ingested as a conventional food containing the same. The health functional food uses food as a raw material, unlike general drugs, thus being advantageous in that there are no side effects that may occur upon administration of the drug for a long time. The content of the active ingredient may be appropriately determined according to the purpose of use (prevention or amelioration), and the active ingredient may be present in a range of 0.1 to 90% by weight with respect to the total weight of the health food composition. However, in the case of long-term intake for health and hygiene purposes or for health control, the amount may be below the above range, or alternatively, the active ingredient may be used in an amount above the range, since there is no problem in terms of safety.

There is no particular limitation as to the type of food. Examples of the food, to which the active ingredient is added, include drinks, meat, sausages, bread, biscuits, rice cakes, chocolate, candy, snacks, confectioneries, pizza, ramen, other noodles, gum, dairy products including ice cream, various soups, beverages, alcoholic beverages, vitamin complexes, dairy products and processed dairy products, and include all health functional foods in the ordinary sense. The morphologies and properties of the food are also not particularly limited, and the food may be provided as any form such as solid, semi-solid, gel, liquid or powder.

A beverage may be prepared using the health functional food composition. There is no particular limitation as to the ingredients of the health drink, other than the active ingredient. The beverage drink may contain, in addition to the active ingredient, various flavoring agents or natural carbohydrates as additives commonly used in the preparation of beverages. The natural carbohydrates may include general sugars, such as monosaccharides (e.g. glucose, fructose, etc.), disaccharides (e.g. maltose, sucrose, etc.) and polysaccharides (e.g., dextrin, cyclodextrin, etc.), as well as sugar alcohols such as xylitol, sorbitol and erythritol. Further, useful flavoring agents may include natural flavoring agents (thaumatin), stevia extracts (rebaudioside A, glycyrrhizin, etc.) and synthetic flavoring agents (saccharin, aspartame, etc.). The natural carbohydrate may be present in an amount of about 1 to about 20 g, preferably about 5 to about 12 g, with respect to 100 mL of the composition of the present invention.

Further, in addition to the active ingredient, the food composition of the present invention may contain other nutrients, vitamins, minerals (electrolytes), flavors such as synthetic or natural flavors, colorants and fillers (cheese, chocolate, etc.), pectic acids and salts thereof, alginic acids and salts thereof, organic acids, protective colloidal thickeners, pH-adjusting agents, stabilizers, preservatives, glycerin, alcohol, carbonic acid used in carbonated beverages, and the like. In addition, it may contain flesh for the production of natural fruit juices, fruit juice beverages and vegetable beverages. These components may be used independently or in combination. Although the proportion of these additives is not so important, the content of these additives is generally selected within the range of 0.1 to 20% by weight in the health functional food composition of the present invention.

### BEST MODE

Hereinafter, the present invention will be described in more detail with reference to the following examples. However, the following examples are provided only for better understanding of the present invention, and thus should not be construed as limiting the scope of the present invention.

### EXAMPLE AND COMPARATIVE EXAMPLE

### Example 1. Synthesis of Aucklandiae Radix Extract

Aucklandiae radix was purchased from the Kyungdong market, and 1 kg of crushed Aucklandiae radix was extracted in 30% ethanol at 80° C. for 5 hours, followed by concentration and freeze-drying to obtain 315 g of an Aucklandiae radix extract (yield 31.5%).

### Examples 2 and 3

Aucklandiae radix extracts were prepared in the same manner as in Example 1, except that the concentration of ethanol during extraction was 50% (Example 2) or 70% (Example 3).

### Comparative Examples 1 and 2

Aucklandiae radix extracts were prepared in the same manner as in Example 1, except that hot-water extraction (Comparative Example 1) and a 95% (Comparative Example 2) ethanol extract were used.

### Experimental Example

### Experimental Example 1: Measurement of Efficacy of Inhibition of Prolactin Secretion

The efficacy of inhibition of prolactin secretion by the Aucklandiae radix extracts obtained in Examples and Comparative Examples (Comparative Examples 1 to 3) were measured using pituitary cells.

Rat pituitary cells GH3 were purchased from the American Type Culture Collection (ATCC), DMEM culture medium, trypsin, and fetal bovine serum (FBS, Gibco™) were purchased from Invitrogen Corporation, and a prolactin ELISA kit for measurement was purchased from Mol-innovation.

GH3 pituitary cells were seeded at a concentration of 4×10⁴ cells/well into a 24-well plate and incubated for 24 hours. In order to evaluate the efficacy of inhibition of prolactin secretion, the cells were treated with 25 and 50 μg/mL of the Aucklandiae radix extracts of Examples 1 to 3 and Comparative Examples 1 to 2 and then incubated for 48 hours, and prolactin secretion was assayed by application to a standard curve using the rat prolactin ELISA kit.

Table 1 shows the effect of inhibition of prolactin secretion by Aucklandiae radix extract.

TABLE 1
Prolactin secretion (ng/mL)

| Sample | 25 μg/mL | 50 μg/mL |
|---|---|---|
| Aucklandiae radix hot water | 1.38 ± 0.216 | 0.52 ± 0.095 |
| extract | | |
| (Comparative Example 1) | | |
| Aucklandiae radix 30% ethanol | 1.17 ± 0.049 | 0.00 ± 0.105 |
| extract (Example 1) | | |
| Aucklandiae radix 50% ethanol | 0.33 ± 0.246 | 0.00 ± 0.056 |
| extract (Example 2) | | |
| Aucklandiae radix 70% ethanol | 1.46 ± 0.047 | 0.00 ± 0.027 |
| extract (Example 3) | | |
| Aucklandiae radix 95% ethanol | 1.02 ± 0.180 | 0.19 ± 0.029 |
| extract (Comparative Example | | |
| 2) | | |

| | |
|---|---|
| Control group | 1 23 ± 0.091 |

As can be seen from Table 1, the Aucklandiae radix exhibited very excellent prolactin secretion inhibitory efficacy in 50 μg/mL of 30% (Example 1), 50% (Example 2) and 70% (Example 3) ethanol extracts, and an animal test of Experimental Example 2 below was performed using the 30% ethanol extract having the lowest alcohol concentration.

### Experimental Example 2: Evaluation of Efficacy of Ameliorating Premenstrual Syndrome in Hyperprolactinemia-Induced Animal Model

Metoclopramide (MCP) is a substance used as a vomiting inhibitor and is known as a drug that increases the secretion of prolactin by inhibiting the secretion of dopamine, and thus inhibits the secretion of progesterone, luteinizing hormone, and follicle-stimulating hormone (Wang et al., 2014). Accordingly, in the present invention, an animal model with premenstrual syndrome induced by hormone secretion disorders was established by administering 20 mg/kg of metoclopramide into the abdominal cavity of female mice every two days for a total of 20 days and then inducing hyperprolactinemia.

ICR mice (female, 12 weeks old) were obtained from Raon Bio Co., Ltd. and were acclimated for 1 week while normal solid feed and water were freely supplied thereto. The environmental conditions of the rearing room were set to a temperature of 23±3° C., a relative humidity of 50±10%, an illumination time of 12 hours (8 am to 8 μm), a ventilation frequency of 10 to 20 times/hour, and illuminance of 150 to 300 Lux. During the experiment period, the temperature and humidity of the rearing room were automatically controlled by a thermo-humidifier.

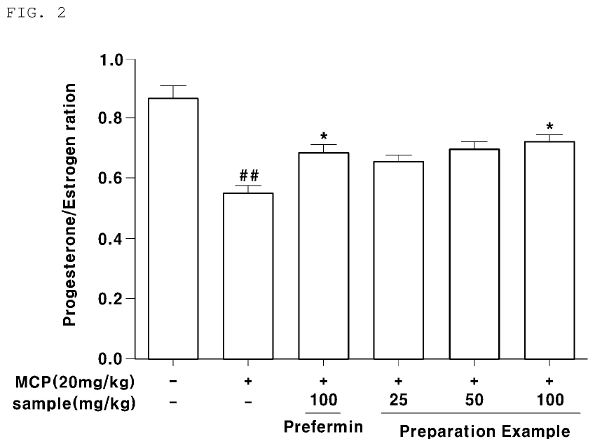

Oral administration was performed on experimental animal groups, namely, 1) a normal group, 2) a hyperprolactinemia-induced control group, 3) a Prefemin-administrated group, 4) a group administered with 25 mg/kg of Preparation Example, 5) a group administered with 50 mg/kg of Preparation Example, and 6) a group administered with 100 mg/kg of Preparation Example. After the end of the experiment, blood was collected, the serum was separated, and the amounts of prolactin, progesterone, estrogen, luteinizing hormone (LH), follicle-stimulating hormone (FSH), and prostaglandin E2 in the serum were measured. The results are schematically shown in FIGS. 2, 3, 4 and 5.

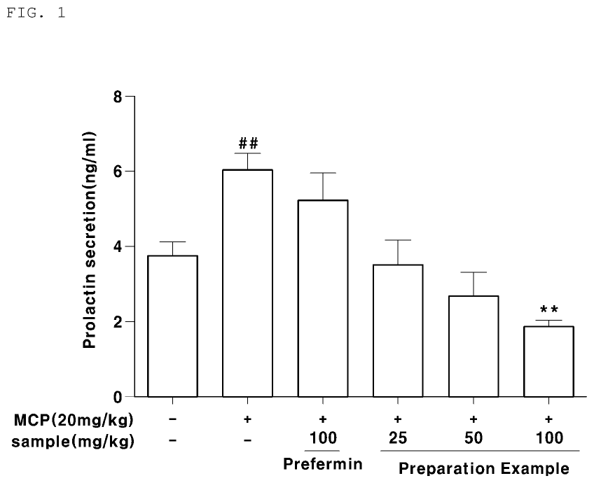

As can be seen from FIG. 1, the content of prolactin in serum was increased due to metoclopramide, and the concentration of prolactin in the blood was effectively reduced by the Aucklandiae radix 30% ethanol extract (Preparation Example) of the present invention.

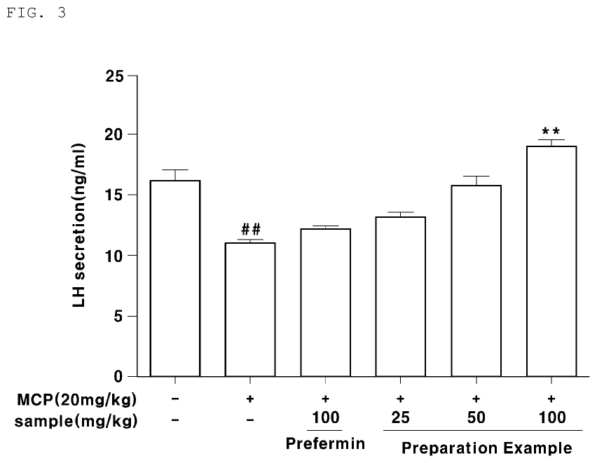

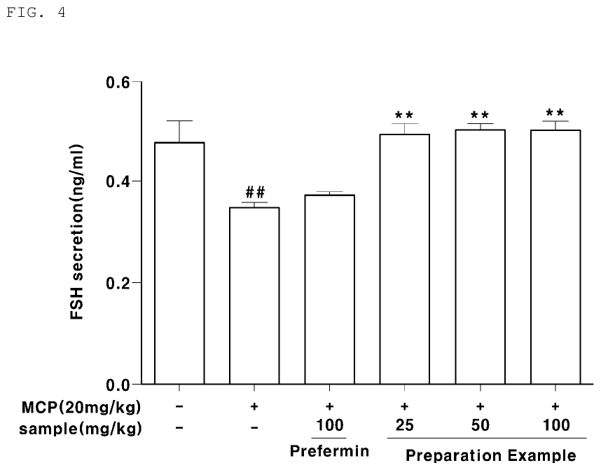

In addition, as can be seen from FIG. 2, the Aucklandiae radix 30% ethanol extract of the present invention effectively increased the secretion of progesterone compared to estrogen. As can be seen from the results of FIGS. 3 and 4, the Aucklandiae radix 30% ethanol extract of the present invention also effectively increased the secretion of luteinizing hormone and follicle-stimulating hormone.

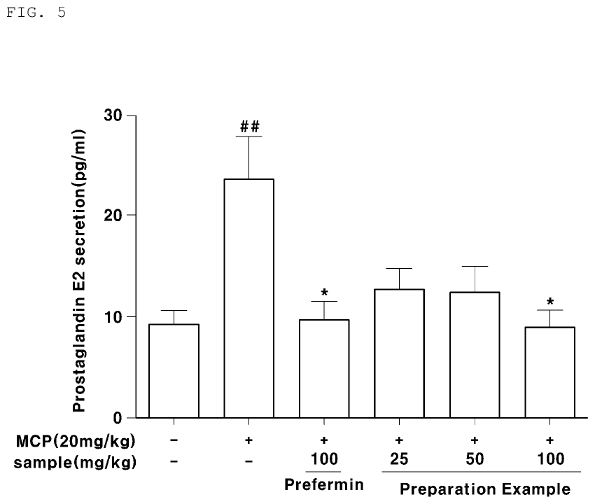

Prostaglandins in blood are substances that regulate various physiological functions such as central nervous system functions, water-electrolyte balance, digestive system functions, and uterus contraction. Deficiency in regulation of blood prostaglandins may cause symptoms of premenstrual syndrome such as affective disorders, headaches, breast pain, bloating, and weight gain. As can be seen from FIG. 5, the Aucklandiae radix 30% ethanol extract of the present invention effectively reduced the concentration of prostaglandin in the blood.

These results indicate that the Aucklandiae radix extract of the present invention inhibits the secretion of prolactin from the pituitary gland cells, thereby increasing deteriorated progesterone secretion occurring in premenstrual syndrome, as well as normalizing the secretion of luteinizing hormone and follicle-stimulating hormone. Based on these functions, the Aucklandiae radix extract is effective as a preventive, ameliorating or therapeutic agent for premenstrual syndrome.

Although embodiments of the present invention have been described with reference to the drawings, it will be obvious to those skilled in the art that the embodiments can be implemented in other specific forms without changing the technical concepts or essential features of the present invention. Therefore, it should be construed that the aforementioned embodiments are illustrative and not restrictive in all respects.

1. A method for preventing, ameliorating or treating premenstrual syndrome of a subject, wherein the method comprises administering an effective amount of Aucklandiae radix extract to the subject in need thereof.

2. The method according to claim 1, wherein the Aucklandiae radix is Aucklandia lappa Decne. or Inula helenium L.

3. The method according to claim 1, wherein the extract is a water extract, a C₁-C₅ alcohol extract, or a C₁-C₅ alcohol aqueous solution extract.

4. (canceled)

5. The method according to claim 1, wherein the Aucklandiae radix extract is a 10% to 80% ethanol aqueous solution extract.

6. The method according to claim 5, wherein the Aucklandiae radix extract is a 20% to 40% ethanol aqueous solution extract.

7. The method according to claim 1, wherein the Aucklandiae radix extract suppresses the secretion of prolactin or increases secretion of progesterone in a woman in a luteal phase before menstruation.

8. The method according to claim 1, wherein a symptom of the premenstrual syndrome comprises at least one of minor psychological discomfort, bloating, weight gain, breast tenderness, muscular tension or aches, poor concentration, and changes in appetite, and the symptom of the premenstrual syndrome appears only in a luteal phase of the menstrual cycle.

9. The method according to claim 1, wherein the Aucklandiae radix extract is administered in a form of a pharmaceutical composition.

10. The method according to claim 1, wherein the Aucklandiae radix extract is administered in a form of a health functional food composition.

11. The method according to claim 8, wherein the Aucklandiae radix extract is present in an amount of 0.001 to 90% by weight based on the total weight of the pharmaceutical composition.

12. The method according to claim 10, wherein the Aucklandiae radix extract is present in an amount of 0.001 to 90% by weight based on the total weight of the health functional food composition.
